(19) United States
(12) Patent Application Publication
(10) Pub. No.: US 2010/0292249 A1
(43) Pub. Date: Nov. 18, 2010

(54) MANUFACTURE OF RIMCAZOLE

(76) Inventor: Ivan Lowdon, Northumberland (GB)

Correspondence Address: MYERS BIGEL SIBLEY & SAJOVEC, PO BOX 37428, RALEIGH, NC, 27627, US

(21) Appl. No.: 12/443,875

(22) PCT Filed: Oct. 1, 2007
(86) PCT No.: PCT/GB2007/003718

§ 371 Date: Jun. 8, 2010

(30) Foreign Application Priority Data
Oct. 2, 2006 (GB) 0619398.1

(51) Int. Cl. A61K 31/496; C07D 403/06

(52) U.S. Cl. 514/254.08; 544/372

(57) ABSTRACT

The present invention relates to a process for preparing Rimcazole and its salts.

The present invention relates to a new improved process for preparing Rimcazole and its salts.

### BACKGROUND

According to European Patent number 0 012 208 B1, the compound named 9-[3-(3,5-cis-dimethylpiperazino)propyl]-carbazole (sometimes named as cis-9-[3-(3,5-dimethylpiperazinyl)propyl]carbazole or 9-[3-(cis-3,5-dimethyl-1-piperazinyl)propyl]carbazole) is useful as a pharmaceutical due to its anti-aggressive and anti-psychotic properties. Herein, we refer to that compound as Rimcazole. Other patent applications, which teach potential uses of Rimcazole include WO/06021811, WO/0000599 and WO/0174359. Rimcazole has the following formula (I):

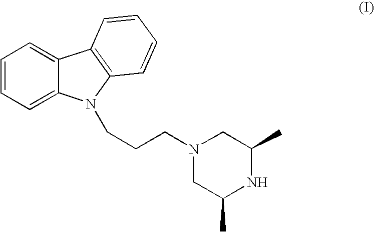

It may be provided in a salt form such as the hydrochloride salt (IA):

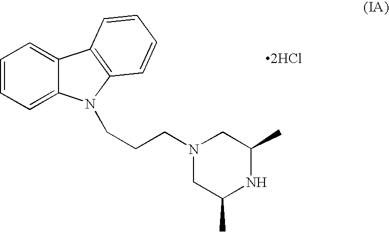

In order to use Rimcazole as a pharmaceutical, large scale quantities, as opposed to small scale laboratory quantities, are required.

One object of the present invention is therefore to provide a new process for the large scale preparation of Rimcazole.

In particular, the present invention seeks to obtain Rimcazole using relatively few chemical reaction steps, from relatively simple starting materials, whilst aiming to avoid isolation of reaction intermediates at completion of each chemical step prior to the start of the next step.

Desirably, the Rimcazole is provided with an acceptable purity for pharmaceutical use using the new process, and this forms a further object of the present invention.

The present invention seeks to achieve the above noted objects by providing a new process for the preparation of Rimcazole as herein explained.

### SUMMARY OF THE INVENTION

According to a first aspect of the present invention there is provided a process for the preparation of a compound of formula (I), including salts thereof, and solvates of salts thereof:

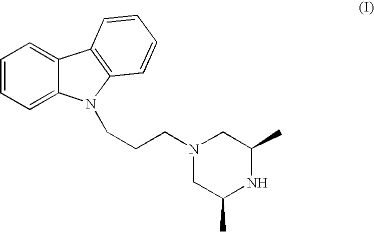

comprising, providing and reacting together a number of compounds in a series of steps i) to iii), according to the following reaction scheme:

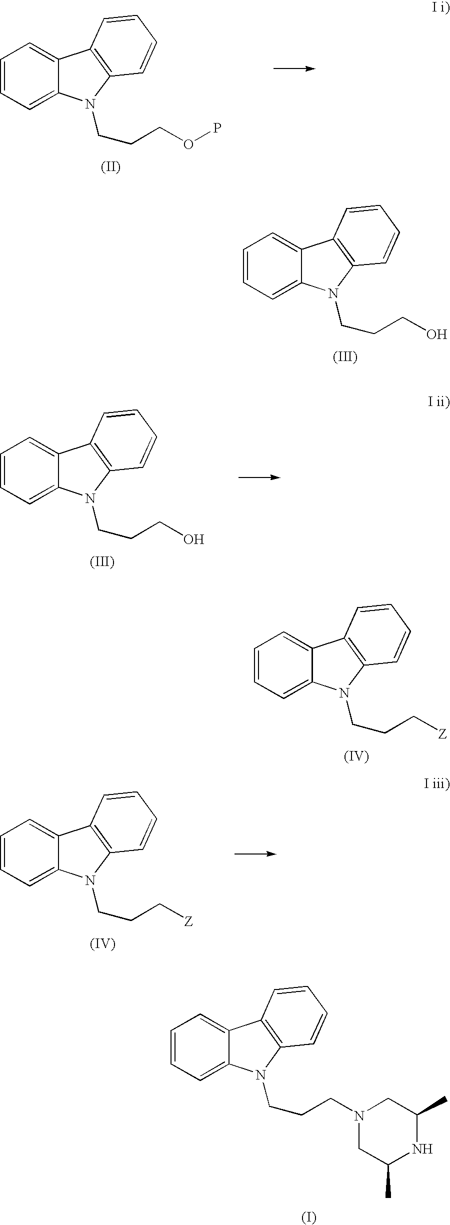

wherein, P is a hydroxyl (—OH) protecting group, and Z is a leaving atom or group.

The hydroxyl protecting group P is preferably an acid labile protecting group, i.e. may be removed by the action of acid, particularly protons (H⁺), to restore the hydroxyl group.

Most preferably, the protecting group P is formed from the reaction of the hydroxyl group with dihydropyran (formula (VII)), as shown generally in the following scheme:

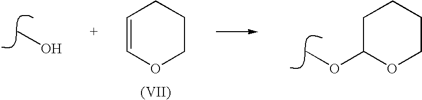

In view of this type of protection, it is preferable to prepare the formula (II) compound, wherein P is a dihydropyran hydroxy protecting group, using a method according to the following scheme:

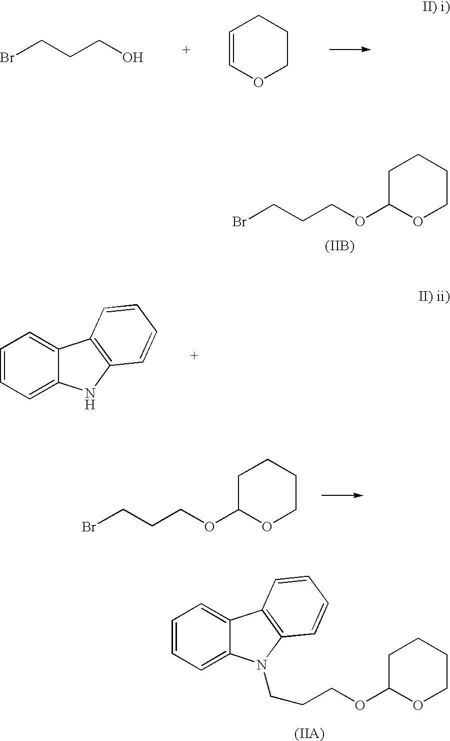

Z may be any atom or group which enables substitution to take place by the amino group at the 4-position of 2,6-cis-dimethypiperazine, which has the following formula:

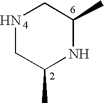

For example, Z may be halo (iodo, bromo, chloro, fluoro, preferably chloro), arylsulphonyloxy (preferably p-toluenesulphonyloxy) or alkylsulphonyloxy (preferably methanesulphonyloxy).

Z is most preferably methanesulphonyloxy.

The use of an acid labile protecting group in the method described herein for the preparation of Rimcazole, and optionally, its salts or solvates of salts, is advantageous in that the alcohol intermediate compound of formula (III) can be prepared in relatively few reaction stages, without extensive purification of reaction products at each stage, and thus provides the intermediate compound (III) in high yield and ready for further reaction by addition of a leaving group Z prior to the final reaction step with 2,6-cis-dimethypiperazine to provide Rimcazole. Further reaction is then available for providing salts of Rimcazole, such as the hydrochloride salt.

Preferably the compound (I) is prepared by incorporating a base wash prior to distillation of the compound, in order to improve stability and/or purity of the compound. Conveniently, the same solvent may be used for stages II i and II ii, which leads to a time saving in the process. The preferred base and solvent for steps II i and II ii are sodium hydroxide and dimethyl formamide (DMF).

Desirably compound IIA is not first purified/isolated, prior to proceeding with step Ii of the process. Preferably methanol and para-toluene sulphonic acid are the reagents used in performing step I i. Compound III is desirably extracted into toluene and washed with aqueous base and water. The toluene layer is thereafter treated with activated carbon and concentrated, before crystalising compound Desirably purity of compound III is >99.5%. If it is not, compound III may be recrystallised from toluene/heptane.

Step I ii is preferably carried out using toluene and methane sulphonyl chloride and triethylamine may be used to remove any hydrochloric acid by-product. Desirably any triethylamine hydrochloride which may be produced, is removed, for example, by one or more water washes.

Compound IA may be formed by convertion of the free base of compound I using extraction into concentrated hydrochloric acid. Isolation of compound IA may be achieved by addition of methylated spirits and any water removed by distillation under vacuum. Crude compound IA may be isolated by filtration and quickly improved by mixing with methylated spirits and drying under vacuum.

For use as a pharmaceutical, the compounds, including physiologically acceptable salts and solvates of the salts, recited herein may be administered singly or in combination with one or more further compounds to a patient in need thereof.

The patient is typically an animal, e.g a mammal, especially a human.

For use according to the present invention, the compounds or physiologically acceptable salts and solvates of the salts, described herein may be presented as a pharmaceutical formulation, comprising the compounds or physiologically acceptable salts and solvates of the salts, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersable granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an

active compound is formulated in an appropriate release—controlling matrix, or is coated with a suitable release—controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

The present invention will now be described with reference to the following non-limiting examples.

### BRIEF DESCRIPTION OF DRAWINGS

The present invention will now be described with reference to the following drawings in which:

FIG. 1 is a ¹H NMR plot of Rimcazole; and

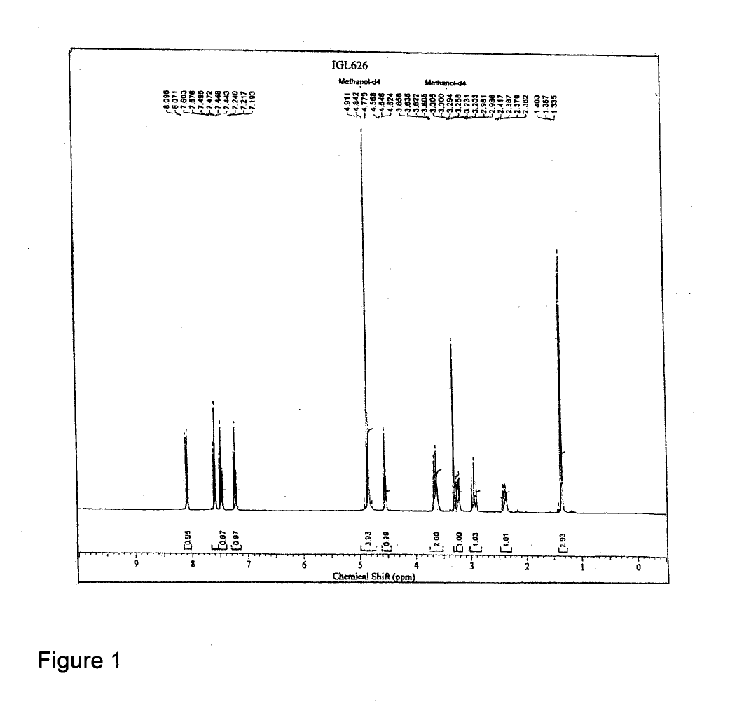

FIG. 2 is a mass spectrum plot of Rimcazole.

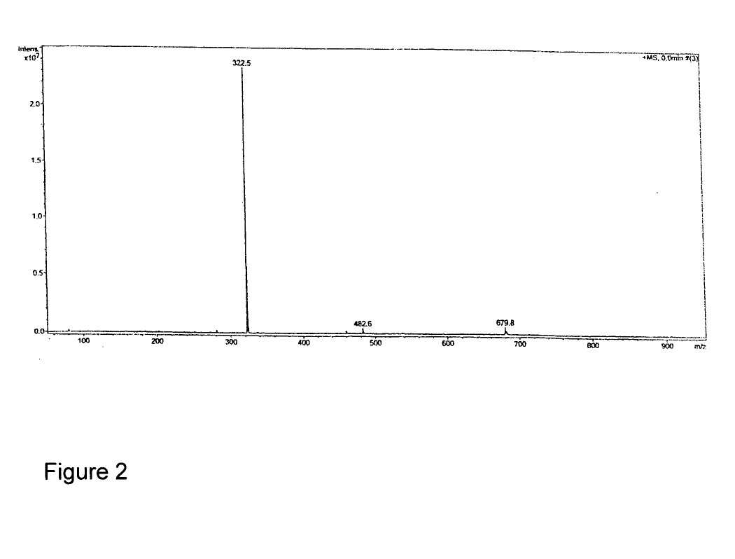

### DETAILED DESCRIPTION

### Compound Synthesis

The di-hydrochloride salt of Rimcazole (IA) as described herein was provided according to the following method, with reference to the following scheme.

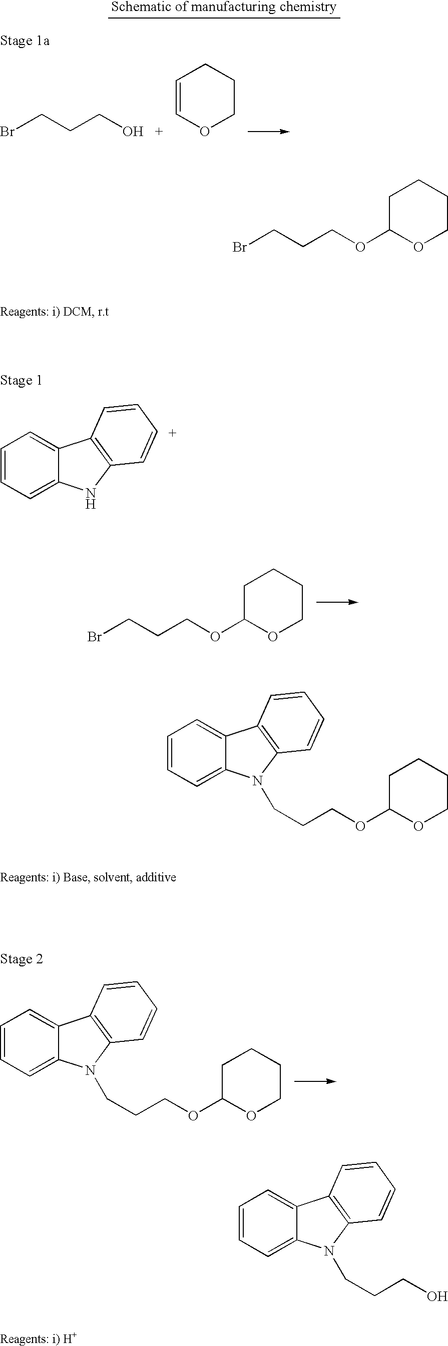

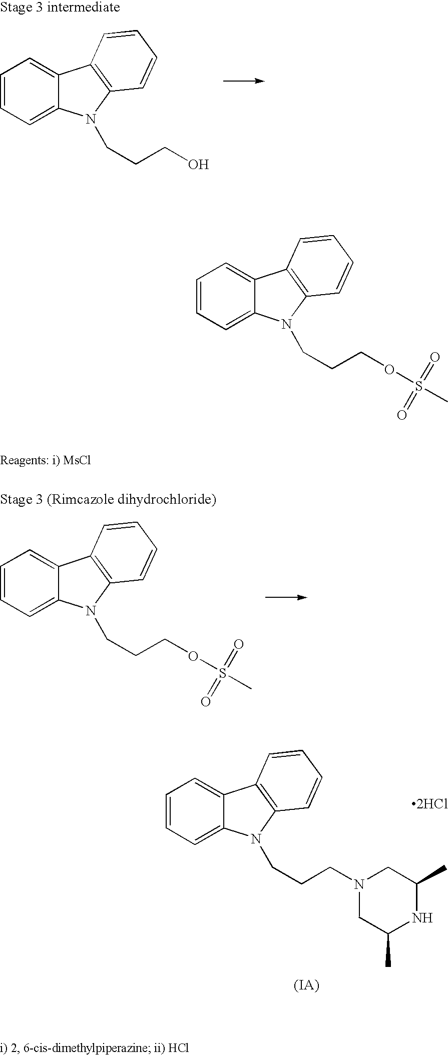

### Process Outlines

| | | | |
|---|---|---|---|
| 1. | Charge 3-Bromo-1-propanol | 2 eq | 400 g |
| 2. | Charge DMF | 5 volumes | 1200 ml |
| 3. | Charge Dihydropyran | 4 eq | 484 g |
| 4. | Stir 1 hour then sample for | | |
| | completion Limit <5% | | |
| | 3-bromo-1-propanol | | |
| 5. | Charge Carbazole | 1eq | 240.6 g |
| 6. | Charge NaOH | 2.0 eq | 115.1 g |
| 7. | Heat to 90° C. and stir until | | |
| | complete Limit <2% carbazole | | |
| 8. | Cool and filter | | |
| 9. | Wash filter cake with DMF | | 200 ml |
| 10. | Charge methanol | 5 volumes | 1200 ml |
| 11. | Charge PTSA | 0.5 eq | 136.8 g |
| 12. | Heat to 50° C. and stir until | | |
| | complete Limit <10% stage one | | |
| 13. | Charge toluene | 5 volumes | 1200 ml |
| 14. | Charge water, stir and separate | 5 volumes | 1200 ml |
| 15 | Extract aqueous with toluene | 5 volumes | 1200 ml |
| 16 | Extract aqueous with toluene | 2.5 volumes | 600 ml |
| 17. | Combine organics and wash with | 5 volumes | 240 g/ |
| | 20% potassium carbonate solution | | 1200 ml |
| 18. | Wash with water | 5 volumes | 1200 ml |
| 19. | Dry over magnesium sulphate | | 400 g |
| 20. | Charge activated carbon | | 30 g |
| 21. | Heat to 90-100° C., stir for | | |
| | 1 hour | | |
| 22. | Filter to remove carbon | | |
| | (hot filtration) | | |
| 23. | Reduce organic volume under vacuum | | |
| | to near dryness | | |
| 24. | Charge toluene | 4.5 volumes | 1080 ml |
| 25. | Charge heptane | 14 volumes | 3360 ml |
| 26. | Heat to form solution | | |
| 27. | Cool and isolate by filtration | | |
| 28. | Wash filter with heptane | 2 volumes | 500 ml |
| 29 | Charge toluene | 4.5 volumes | 1080 ml |
| 30. | Charge heptane | 14 volumes | 3360 ml |
| 31. | Heat to form solution | | |
| 32. | Cool and isolate by filtration | | |
| 33. | Wash filter with heptane | 2 volumes | 500 ml |

Yield: 191.8 g (59%)

Specification: Material must be >99.5% pure by HPLC, if not repeat steps 29-33. A 90% recovery is expected for each recrystallisation.

Stage 3

| 1. | Charge stage 2 | 1 | eqs | 155 | g |
|---|---|---|---|---|---|
| 2. | Charge toluene | 10 | vols | 1550 | ml |
| 3. | Charge triethylamine | 3 | eqs | 209 | g |
| 4. | Heat to form a | | | | |
| | solution >35° C. | | | | |
| 5. | Charge methane | 1.05 | eqs | 78.8 | g |
| | sulphonyl | | | | |
| | chloride at 35-40° C. | | | | |
| 6 | Stir until complete | | | | |
| | (add more | | | | |
| | MsCl if req.) Limit < | | | | |
| | 1% stage | | | | |
| | two | | | | |
| 7. | Charge water | 5 | vols | 775 | ml |
| 8. | Wash with 10% NaCl | 5 | vols | 77.5 g/775 ml | |
| | solution | | | | |
| 9 | Dry over magnesium | | | 50 | g |
| | sulphate | | | | |
| 17. | Wash with 10% NaCl | 5 | vols | 77.5/775 ml | |
| | solution | | | | |
| 18. | Concentrate organic to near | | | | |
| | dryness under vacuum | | | | |
| | at 50° C. | | | | |
| 19. | Charge toluene | 10 | vols | 1550 | ml |
| 20. | Concentrate organic to near | | | | |
| | dryness under vacuum | | | | |
| | at 50° C. | | | | |
| 21. | Charge toluene | 10 | vols | 1550 | ml |
| 22. | Analysis organic layer for | | | | |
| | Triethylamine content, if >1% | | | | |
| | repeat steps 20-21 until <1%. | | | | |
| 23. | Charge conc HCl | 6 | eqs | 465 | ml |
| 24 | Separate, collecting lower | | | | |
| | product layer | | | | |
| 25. | Charge IMS | 20 | vols | 3100 | ml |
| 26. | Concentrate organic to near | | | | |
| | dryness under vacuum | | | | |
| | at 50° C. | | | | |
| 27. | Charge IMS | 20 | vols | 3100 | ml |
| 28. | Concentrate organic to | | | | |
| | near dryness under | | | | |
| | vacuum at 50° C. | | | | |
| 29. | Charge IMS, stir for 30 | 20 | vols | 3100 | ml |
| | minutes at room temp | | | | |
| 30. | Filter and wash with IMS | 2 | vols | 310 | ml |
| 31. | Recharge solid | | | | |
| 32. | Charge IMS | 12 | vols | 1860 | ml |
| 33. | Heat to reflux and stir 1 hour | | | | |
| 34. | Cool to 15-25° C. and stir | | | | |
| | for at least 1 hour | | | | |
| 35. | Filter and wash with IMS | 2 | vols | 310 | ml |
| 36. | Dry to constant weight | | | | |
| | under vacuum at 45-55° C. | | | | |

| | | | |
|---|---|---|---|
| Overall yield: | 188.6 | g | (69.5%) |

Control of Materials

All materials used in the development work were standard grade laboratory reagents purchased from a variety of suppliers.

For GMP manufacture a specification and analytical monograph will be set for each raw material.

Control of Critical Steps and Intermediate

Reaction completions and isolated intermediates are controlled by HPLC analysis using the analytical method described in section S.3.1.

Process Validation and/or Evaluation

No process validation or evaluation has occurred.

Manufacturing Process Development

The selected route was initially run on a small scale, as this successfully produced the desired product the route was deemed the best option and no alternative routes were examined.

Initially the stage 1a intermediate was produced in a separate stage and attempts were made to isolate this in a high purity using distillation. This showed that the stage 1a material was actually unstable to distillation due to residual acidity, which led to deprotection of the material upon stressing. This could be solved by incorporating a base wash into the process prior to distillation but it was found that fractional distillation was required to achieve a chemical purity of >95%.

It was found that the stage 1a could be performed in the reaction solvent used for the stage 1 chemistry and this has been selected as the chosen method. This gives a slightly lower yield at stage 2 but the saving in processing time more than compensates for this.

A variety of alternative bases and solvents were examined for the stage 1 chemistry and it was found that sodium hydroxide with DMF as solvent was the best method. It was found that the level of carbazole in the reaction completion is important as at a level of >2% there is the possibility that the material is carried through to the isolated stage two. Therefore a completion limit of <2% carbzole has been set.

Isolation of the stage one was found to be difficult and as the material can again be used insitu for the stage 2 reaction this option was not pursued.

Methanol and para-toulene sulphonic acid were found to be the best reagents for performing the stage 2 deprotection reaction. It was found that this reaction is in fact in an equilibrium state and as such a low level of stage one in the reaction completion cannot always be achieved. Therefore the reaction completion is set at <10% stage one and the work up removes this material. There is evidence that the reverse reaction can occur during the isolation of the product. This is due to residual acidity and a base wash has been incorporated to prevent this.

Initial attempts to isolate the stage 2 product focused on using a simple quench of the DMF reaction mixture into water and subsequent isolation of the solid produced. Unfortunately although this works on a small laboratory scale the method is not suitable for large-scale manufacture due to the form of the slurry produced.

Development has produced a method whereby the product is extracted into toluene, washed with aqueous base then water. The toluene layer is treated with activated carbon to remove trace impurities and is then concentrated. The stage 2 is recrystallised from a mixture of toluene and heptane. The quality of stage two was found to be critical, as the purity of the stage two has a direct impact on the purity at stage 3. A purity of at least 99.5% is required and this can be achieved by re-crystallising the material from toluene/heptane.

The stage 3 mesylation reaction is performed using toluene and methane sulphonyl chloride. Triethylamine is used as a base to remove the hydrochloric acid that is produced as a by-product of the reaction. The methane sulphonyl chloride charge is kept to a minimum in order to prevent any possible side reactions that could occur if an excess is used. The reaction is monitored by HPLC and additional methane sulphonyl chloride added if required to achieve the desired completion.

It was found that the triethylamine hydrochloride produced must be removed using water washes otherwise this can lead to the formation of impurities during the stage 3 chemistry.

The intermediate mesylate has been found to be very stable as experimentation has shown that the material can be azeotroped dry at high temperatures. The water content on the mesylate is not critical as the reaction with cis-dimethyl piperazine does proceed in the presence of water, but laboratory work indicates that a high water content can lead to increased levels of the typical impurities. Although these are removed by the work up method it is advisable to dry the mesylate.

The stage three coupling to cis-dimethyl piperazine was found to work well but an additional charge of triethylamine is required to ensure that the methane sulphonic acid produced is removed as this can also lead to impurities being formed. A sodium hydroxide treatment is used to ensure that none of the product exists as a salt as the pKA values of the Rimcazole free-base and triethylamine are similar which could mean that the methane sulphonic acid salt of Rimcazole is formed. Aqueous washes are used to remove inorganics.

The toluene layer is then concentrated to remove residual triethylamine as this can interfere with the formation of the dihydrochloride salt. Development is looking at using GC analysis to monitor the triethylamine level and to set an appropriate limit.

It was found that the mono-hydrochloride salt is very insoluble in both common organic solvents and water. As the more soluble (in water) di-hydrochloride salt was required the method whereby the salt is produced by gassing the organic layer with HCl gas has not been used, as this yielded the mono-hydrochloride salt.

A method whereby the free base is converted to the di-hydrochloride salt using extraction into concentrated hydrochloric acid has been developed. The material is then isolated by the addition of industrial methylated spirits and removal of the water by distillation. The work has shown that this distillation must be performed under vacuum as atmospheric distillation can lead to the formation of an impurity that cannot be removed easily. The crude product is isolated by filtration then re-slurried in hot industrial methylated spirits which improves the product quality.

The material is dried under vacuum to a constant weight. No evidence of hydroscopsity, often seen in HCl salts has been observed in the laboratory.

Typically this process produces material at >99% chemical purity by HPLC analysis with four impurities all at less than 0.5%.

Characteristation

Elucidation of Structure and Other Characteristics

A sample of authentic Rimcazole was purchased from the Aldrich Chemical Company and this was used as a reference for confirmation of structure by ¹H NMR and HPLC.

Mass spectrum also confirms the desired mass ion to be present.

The melting point of material produced and described herein was found to correspond well to the literature value.

The HCl content was determined using a titration method and results indicate the di-hydrochloride salt.

### Analysis

Weigh out accurately approximately 1 g sample into a 250 ml conical flask. Dissolve in 50 ml methanol and add 10 ml glacial acetic acid. Titrate against 0.1M silver nitrate using Eosin A as indicator.

### Calculation:

$$Number of mols C {l}^{-} = \frac{Titre ( ml ) \times Molarity of silver nitrate}{1000}$$

$$Mass of H C l ( g ) = mols of {Cl}^{-} \times R M M$$

$$\% w \text{/} w of H C l in sample = \frac{Mass of H C l ( g ) \times 100}{Sample weight ( g )}$$

### Typical Analysis

| Test | Result | |
|---|---|---|
| APPEARANCE | An off-white solid. Free from | |
| | visible contamination. | |
| IDENTITY BY ¹H-NMR | Conforms to structure. | |
| IDENTITY BY MS | Conforms to structure. | |
| MELTING POINT | Discoloured at 270° C. | |
| | Melt at 290° C. | |
| HCl CONTENT | 17.81% w/w. | |
| CHEMICAL PURITY BY HPLC | 99.56% | |
| RESIDUE ON IGNITION | None detected. | |
| RESIDUAL SOLVENTS BY HRGC | Methanol | None detected. |
| | Ethanol | 0.17% w/w. |
| | Dichloromethane | LT0.01% w/w. |
| | Heptane | LT0.01% w/w. |
| | Toluene | 0.01% w/w. |
| | DMF | LT0.01% w/w. |

1. A method for the preparation of a compound according to formula (I), a salt thereof or a solvate of a salt thereof: 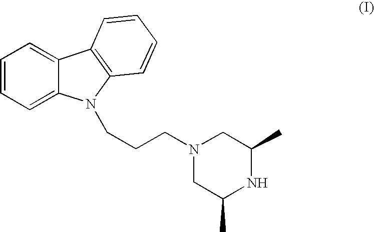
comprising, providing a compound according to formula (II): 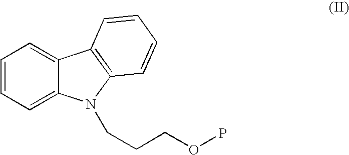
wherein P is a hydroxyl protecting group; deprotecting the compound of formula (II) to form a compound of formula (III): 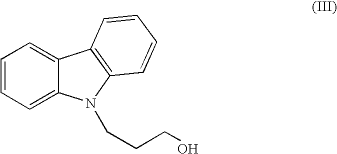
reacting the compound of formula (III) to form a compound of formula (IV): 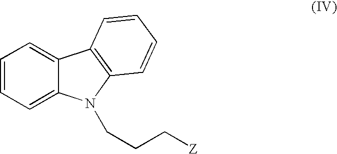
wherein Z is a leaving atom or group;
and reacting the compound of formula (IV) with 2,6-cis-dimethypiperazine to form the compound of formula (I).

2. The method according to claim 1 wherein P is an acid labile protecting group.

3. A method according to claim 1, wherein the compound according to formula (II) has a structure of formula (IIA):

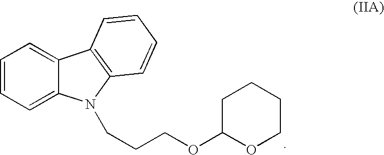

4. A method according to claim 3, wherein the compound of formula (IIA) is prepared by reacting a compound of formula (IIB): 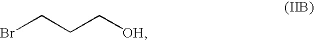
with a compound of formula (IIC): 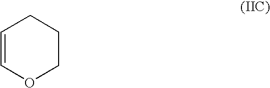
to provide a compound of formula (IID): 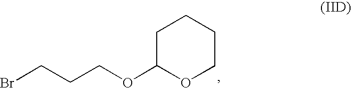
and subsequent reaction with a compound of formula (IIE):

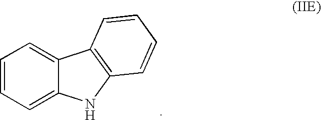

5. The method according to claim 1 wherein Z is halo (iodo, bromo, chloro, fluoro, preferably chloro), arylsulphonyloxy (preferably p-toluenesulphonyloxy) or alkylsulphonyloxy (preferably methanesulphonyloxy).

6. The method according to claim 5 wherein Z is methanesulphonyloxy.

7. The method according to claim 1 further comprising reacting the compound of Formula (I) with an acid in order to form a salt of Formula (I).

8. The method according to claim 7 wherein the acid is hydrochloric acid.

9. The method according to claim 1 wherein all steps required for obtaining the compound according to claim (IV) are carried out using the same solvent.

10. The method according to claim 9 wherein the solvent is dimethyl formamide.

11. The method according to claim 3
wherein compound IIA is not first purified and/or isolated prior to proceeding with the next step.

12. The method according to claim 1 wherein compound (I) or a salt thereof, or a solvate of a salt thereof, is produced at a purity of ≧99%.

13. A pharmaceutical formulation comprising a compound according to formula (I) or a salt thereof or a solvate of a salt thereof, as prepared according to claim 1, together with a pharmaceutically acceptable salt thereof.
